(19) United States
(12) Patent Application Publication
(10) Pub. No.: US 2021/0259233 A1
(43) Pub. Date: Aug. 26, 2021

(54) FOGGER MACHINE

(71) Applicant: THE FOUNTAINHEAD GROUP, INC., New York Mills, NY (US)

(72) Inventor: Matthew Merritt, Oneida, NY (US)

(73) Assignee: THE FOUNTAINHEAD GROUP, INC., New York Mills, NY (US)

(21) Appl. No.: 17/181,333

(22) Filed: Feb. 22, 2021

Related U.S. Application Data
(60) Provisional application No. 62/979,809, filed on Feb. 21, 2020.

(51) Int. Cl. A01M 1/20

(52) U.S. Cl. CPC A01M 1/2072

(57) ABSTRACT

A fogging machine for emitting an insecticide in a fog or mist is provided. The fogger machine is powered by a rechargeable power source that is coupled to a solar panel that collects and converts sunlight into electrical energy. A microprocessor can be programmed, configured and/or structured to permit the machine to be turned on-off at preselected times. A Bluetooth or other wireless communication circuit permits the unit to be controlled remotely via a smart phone/computer/tablet.

### CROSS-REFERENCE TO RELATED APPLICATION

This application relates and claims priority to U.S. Provisional Patent Application No. 62/979,809, filed with the USPTO on Feb. 21, 2020, the disclosures of which is incorporated herein by reference in its entirety.

### FIELD OF THE INVENTION

The present disclosure is directed generally to fogger machines used to emit a fog or mist of insecticide or similar chemical.

### BACKGROUND

Foggers are commonly used in an outdoor and sometimes an indoor environment to emit a fine mist or fog that contains chemicals to kill or repel mosquitos and other bugs/insects. Thermal foggers use a heating element that heats the chemical prior to its dispersion. Most foggers are powered by propane, battery, or AC electrical power. A user can place a fogger in a desired location and manually turn the fogger and let it run for a predetermined time period before turning it off. After some time-lapse to allow the chemicals to disperse and become diluted through the air, the area impacted by the fogger is then safe for people and should be free from the mosquitos or other insects the chemical was intended to kill.

It would be useful to have a fogger that could automatically turn on and off at predetermined intervals and otherwise automate the process associated with fogging.

Accordingly, there is a need in the art for a fogger equipped with automatically rechargeable power supply, and communication capabilities that permit remote operation of the fogger machine.

### SUMMARY

The present disclosure is directed to a fogger machine.

According to an aspect is a fogger machine, comprising a housing having an inlet and an outlet arranged to permit 360 degree output; a fan positioned adjacent the inlet and arranged to pull air into and through the housing; a rechargeable battery power supply; a replaceable chemical cartridge; a solar cell operably connected to the battery power supply; and a microprocessor configured, programmed and/or structured to turn the power supply on and off at predetermined intervals.

According to an embodiment, the fogger machine further comprises a sensor to detect the level of chemical left in the chemical cartridge.

According to an embodiment, the fogger machine further comprises a user alert operably associated with the sensor to provide the user with indication as to the level of chemical left in the cartridge.

According to an embodiment, wherein the user alert can comprise at least one of an LED indicator and a Bluetooth Alert.

According to an embodiment, the fogger machine further comprises wherein the sensor can be at least one of an electrical sensor, a mechanical sensor, and an electro-mechanical sensor.

According to an embodiment, the fogger machine further comprises a stake to mount the fogger machine in a desired location.

According to an embodiment, wherein the microprocessor is configured, programmed, and/or configured to receive signals from the solar cell indicative of the time of day.

According to an embodiment, the fogger machine further comprises a heating element that is adapted to vaporize the chemicals in the chemical cartridge with the fan blowing or drawing air past the heating element.

These and other aspects of the invention will be apparent from the embodiments described below.

### BRIEF DESCRIPTION OF THE DRAWINGS

The present invention will be more fully understood and appreciated by reading the following Detailed Description in conjunction with the accompanying drawings, in which:

FIG. 1 is a perspective view of a fogger machine, in accordance with an embodiment.

FIG. 2 is a perspective view of the top of the fogger machine, in accordance with an embodiment.

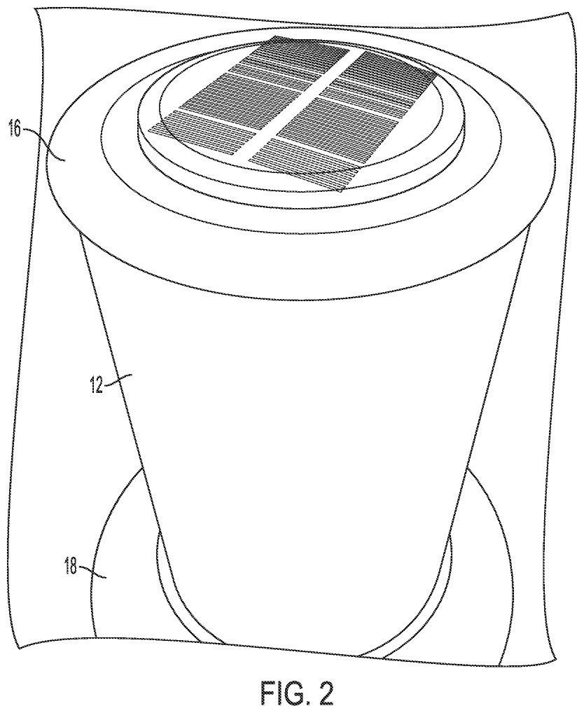

FIG. 3 is a bottom perspective view of the fogger machine, in accordance with an embodiment.

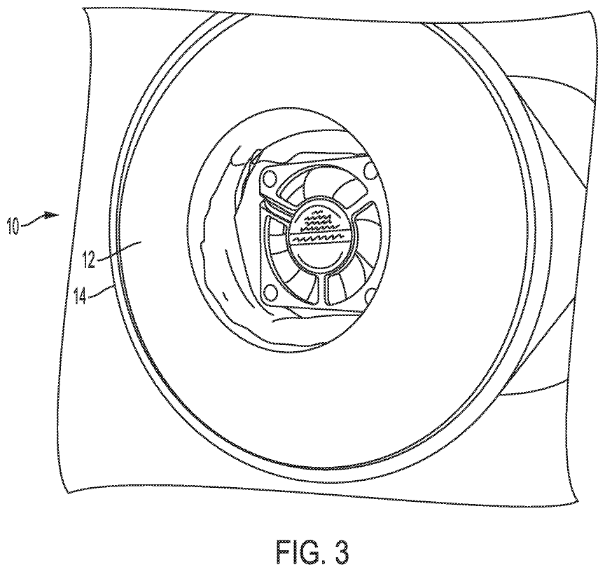

FIG. 4 is an enlarged view of the top of the fogger machine, in accordance with an embodiment.

FIG. 5 is a cross-sectional view taken along section line 4-4 of FIG. 1 of the fogger machine, in accordance with an embodiment.

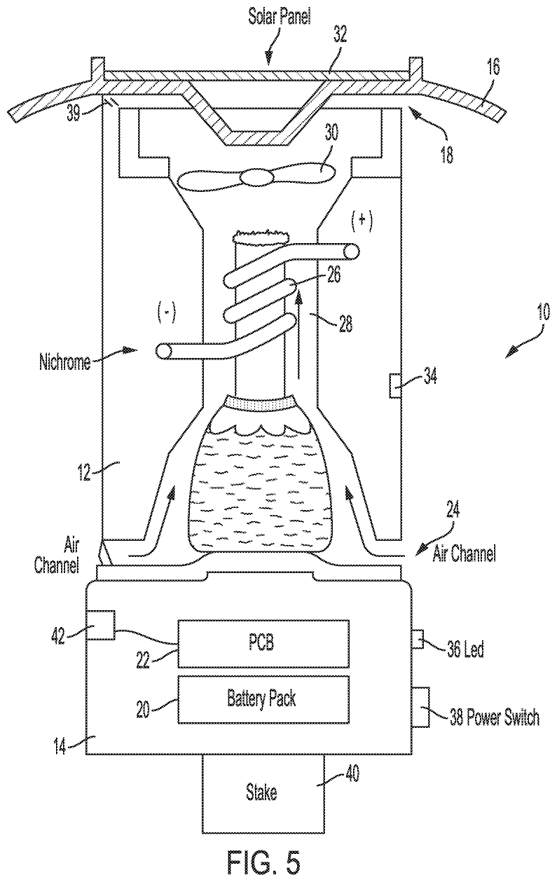

### DETAILED DESCRIPTION OF EMBODIMENTS

The present disclosure describes a fogger machine 10. Fogger machine 10 is adapted to generate a fog or mist that contains a chemical, such as an insecticide, to kill or repel insects, such as mosquitos, and it can function as a mister, a thermal fogger, cold fogger, auto-fogger, or manual fogger.

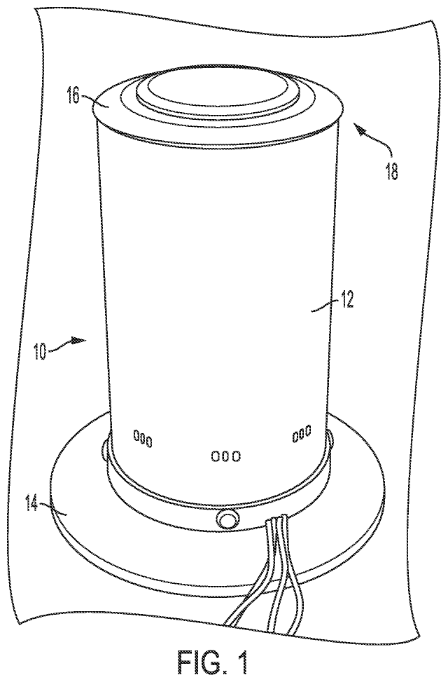

Referring to FIG. 1, in one embodiment, is fogger machine 10 that generally comprises a housing 12, preferably cylindrical in shape, a base 14, and a top 16 that is attached to in slightly vertically spaced relation to housing 12. A gap 18 is formed circumferentially around (360 degrees) in the space between housing 12 and top 16. Fog generated by fogging machine 10 is emitted 360 degrees around fogger machine 10 through gap 18.

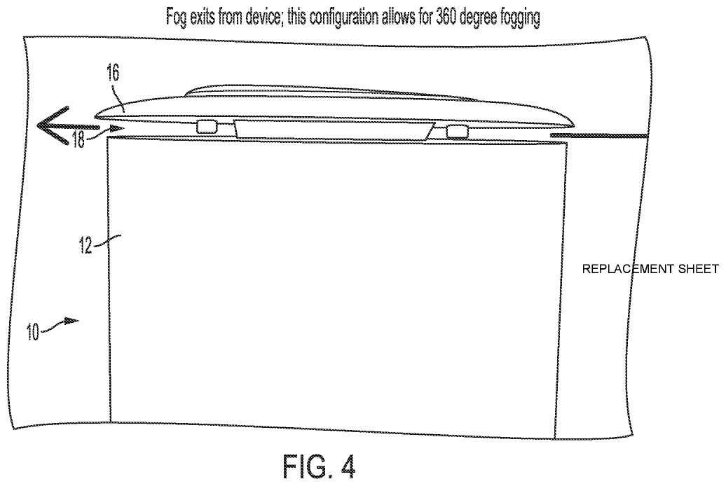

As best seen in FIG. 4, housed within fogger machine 10 are a power source 20, preferably a rechargeable battery, a microprocessor/printed circuit board 22 that receives power from power source 20, an air inlet 24 formed between base 14 and housing 12 into which air is drawn into and through housing 12, a thermal component 26 (e.g., NiChrome flat wire) that heats the chemical contained within chemical cartridge 28 also mounted within housing 12, a fan 30 used to pull air into, through, and out of housing 12, and a solar panel 32 mounted atop top 16 that collects solar energy and converts it into electrical energy used to charge power source 20. A chemical level sensor 34 is provided to sense the quantity of chemical left in cartridge 28 and when dropped below a predetermined threshold, provide a signal to actuate an alert 36 that cues the user that the chemical level has dropped below a threshold. The sensor 34 can be an electrical, mechanical, or electro-mechanical sensor, and the alert 36 can be a visual alert such as an LED, an audible alert, or some combination thereof. A power switch 38 is also provided to turn fogger machine 10 on or off.

While fogger machine 10 is shown as being powered by a solar rechargeable battery, an optional AC electrical power source could also be provided to permit the unit to be used in low-sun areas or indoors.

Upon actuation, thermal component 26 heats up, fan 30 draws air into and through housing 12 where it is mixed with chemical dispersed from cartridge 28, and then emitted as a mist or fog through gap 18.

Gap 18 could include louvers or other air blocking elements to direct the mist/fog in a desired direction if less than 360-degree emission is desired. In addition, a stake 40 is provided that permits the fogger 10 to be set into the ground in a desired location; otherwise the unit can be set on base 14 in a desired location.

A wireless/Bluetooth circuit 42 can also be included to permit the unit to be controlled remotely via an app from a smartphone/computer/tablet.

The user can configure the microprocessor 22 in order set the fogger machine 10 to turn on and off at set times of the day. Also, microprocessor 22 can learn daylight, dusk, and sunrise times via feedback provided by solar panel 32. Using this feedback and time learning functionality, the unit could be set to turn on when mosquito activity is heightened (i.e., during dusk hours).

Fogger machine 10 can be constructed and gasketed with weatherproof and rust-proof materials.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The above-described embodiments of the described subject matter can be implemented in any of numerous ways. For example, some embodiments may be implemented using hardware, software or a combination thereof. When any aspect of an embodiment is implemented at least in part in software, the software code can be executed on any suitable processor or collection of processors, whether provided in a single device or computer or distributed among multiple devices/computers.

What is claimed is:

1) A fogger machine, comprising:
a) a housing having an inlet and an outlet arranged to permit 360-degree output;
b) a fan positioned adjacent the inlet and arranged to pull air into and through the housing;
c) a rechargeable battery power supply;
d) a replaceable chemical cartridge;
e) a solar cell operably connected to the battery power supply; and
f) a microprocessor configured, programmed and/or structured to turn the power supply on and off at predetermined intervals.

2) The fogger machine according to claim 1, further comprising a sensor to detect the level of chemical left in the chemical cartridge.

3) The fogger machine according to claim 2, further comprising a user alert operably associated with the sensor to provide the user with indication as to the level of chemical left in the cartridge.

4) The fogger machine according to claim 3, wherein the user alert can comprise at least one of an LED indicator and a Bluetooth Alert.

5) The fogger machine according to claim 2, wherein the sensor can be at least one of an electrical sensor, a mechanical sensor, and an electro-mechanical sensor.

6) The fogger according to claim 1, further comprising a stake to mount the fogger machine in a desired location.

7) The fogger machine according to claim 1, wherein the microprocessor is configured, programmed, and/or configured to receive signals from the solar cell indicative of the time of day.

8) The fogger machine according to claim 1, further comprising a heating element that is adapted to vaporize the chemicals in the chemical cartridge with the fan blowing or drawing air past the heating element.
